(19) United States
(12) Patent Application Publication
(10) Pub. No.: US 2024/0293614 A1
(43) Pub. Date: Sep. 5, 2024

(54) AGENT ADMINISTERING MEDICAL DEVICE

(71) Applicant: Boston Scientific Scimed, Inc., Maple Grove, MN (US)

(72) Inventors: Matthew Robert JAGELSKI, Marlborough, MA (US); Andrew PIC, Northboro, MA (US); Amanda Lynn SMITH, Boston, MA (US); Ra NAM, Lawrence, MA (US); Laurie A. LEHTINEN, Boylston, MA (US)

(73) Assignee: Boston Scientific Scimed, Inc., Maple Grove, MN (US)

(21) Appl. No.: 18/586,812

(22) Filed: Feb. 26, 2024

Related U.S. Application Data
(63) Continuation of application No. 17/108,495, filed on Dec. 1, 2020, now Pat. No. 11,918,780.
(60) Provisional application No. 62/942,988, filed on Dec. 3, 2019.

(51) Int. Cl. A61M 5/155; A61M 5/145; A61M 5/315

(52) U.S. Cl. CPC A61M 5/155; A61M 5/1454; A61M 5/31586; A61M 2202/0225; A61M 2210/0625; A61M 2210/105; A61M 2210/1053; A61M 2210/1067

(57) ABSTRACT

A medical device comprising an enclosure defining a cavity for containing agent, a lumen for receiving a pressurized gas, and a barrier positioned between the cavity and the lumen, the barrier including at least one opening for storing agent, wherein rotation of the barrier relative to the lumen establishes fluid communication between the at least one opening and the lumen for delivering agent from the at least one opening to the lumen.

### CROSS-REFERENCE TO RELATED APPLICATIONS

This application claims the benefit of priority from U.S. Provisional Application No. 62/942,988, filed on Dec. 3, 2019, which is incorporated by reference herein in its entirety.

### TECHNICAL FIELD

The present disclosure relates generally to a medical device that administers an agent. More particularly, at least some embodiments of the present disclosure relate to a medical device including a system that can be actuated to administer a dosage of an agent to a lumen.

### BACKGROUND

In certain medical procedures, it may be necessary to stop or minimize bleeding internal to the body. For example, an endoscopic medical procedure may require hemostasis of bleeding tissue within the gastrointestinal tract, for example in the esophagus, stomach, or intestines.

During an endoscopic procedure, a user inserts a sheath of an endoscope into a body lumen of a patient. The user utilizes a handle of the endoscope to control the endoscope during the procedure. Tools are passed through a working channel of the endoscope via, for example, a port in the handle, to deliver treatment at the procedure site near a distal end of the endoscope. The procedure site is remote from the operator.

To achieve hemostasis at the remote site, a hemostatic agent may be delivered by a device inserted into the working channel of the endoscope. Agent delivery may be achieved through mechanical systems, for example. Such systems, however, may require numerous steps or actuations to achieve delivery, may not achieve a desired rate of agent delivery or a desired dosage of agent, may result in the agent clogging portions of the delivery device, may result in inconsistent dosing of agent, or may not result in the agent reaching the treatment site deep within the GI tract. The current disclosure may solve one or more of these issues or other issues in the art.

### SUMMARY OF THE DISCLOSURE

According to an example, a medical device may comprise an enclosure defining a cavity for containing agent, a lumen for receiving a pressurized gas, and a barrier positioned between the cavity and the lumen, the barrier including at least one opening for storing agent, wherein rotation of the barrier relative to the lumen establishes fluid communication between the at least one opening and the lumen for delivering agent from the at least one opening to the lumen. A bottom end of the cavity may include a wall adjacent to the barrier, wherein the wall includes a wall opening, wherein the wall opening is located on an area of the wall so that the wall opening is aligned with the at least one opening via rotation of the barrier. Alignment of the wall opening with the at least one opening may permit agent from the enclosure to enter the at least one opening. The enclosure may be rotatable relative to the barrier and/or the lumen. The barrier may be rotatable relative to the enclosure. The agent may remain in the at least one opening until fluid communication between the at least one opening and the lumen is established. The enclosure may feed the at least one opening with agent via gravity. The at least one opening for storing agent may feed the lumen with agent via gravity when fluid communication between the at least one opening and the lumen is established.

In another example, the at least one opening for storing agent may be a plurality of openings arranged radially about the barrier. The plurality of openings may be symmetrically arranged. Each of the plurality of openings may be different in size. When fluid communication is established between one opening of the plurality of openings and the lumen, the other openings of the plurality of openings and the lumen are not in fluid communication.

In another example, the medical device may further comprise an intermediary barrier, wherein the intermediary barrier is positioned between the barrier and the lumen, and wherein the intermediary barrier includes an intermediary opening positioned to be aligned with one of the plurality of openings via rotation of the barrier. The intermediary barrier may be rotatable relative to the barrier. The lumen may be a flexible catheter capable of traversing a tortuous body lumen, and further comprising a source of the pressurized gas.

According to an example, a medical device may comprise a cartridge including a plurality of chambers, wherein each of the chambers stores a pre-filled amount of agent, a lumen for receiving a pressurized gas, a channel establishing fluid communication between a first end of the cartridge and the lumen for delivering agent from the cartridge to the lumen, and a plunger coupled to a second end of the cartridge so that the plunger is aligned with one chamber of the plurality of chambers, wherein the plunger advances longitudinally into the one chamber, thereby pushing the pre-filled amount of agent towards the channel, and wherein the cartridge is rotatable relative to the plunger to align the plunger with another of the plurality of chambers. The plunger may be coupled to the cartridge so that the plunger is spring-biased to a position outside of the one chamber and aligned with the one chamber. The medical device may further comprise a trigger including a lever coupled to a linkage via a first articulating joint and a linkage coupled to a plunger via a second articulating joint.

According to an example, a method of administering an agent via a medical device may comprise positioning the medical device, including an enclosure, a barrier, and a lumen, so that a distal end of the lumen is adjacent to a targeted site, wherein the barrier is positioned between the enclosure and the lumen, the enclosure containing agent, and the barrier including at least one opening for storing the agent, providing a pressurized gas to the lumen, and rotating the barrier relative to the lumen so that fluid communication is established between the at least one opening and the lumen to deliver the agent from the at least one opening to the lumen. The method may further comprise rotating the barrier relative to the lumen so that the at least one opening and the lumen are not in fluid communication after a dose of the agent is delivered from the at least one opening to the lumen.

### BRIEF DESCRIPTION OF THE DRAWINGS

The accompanying drawings, which are incorporated in and constitute a part of this specification, illustrate various exemplary embodiments and together with the description, serve to explain the principles of the disclosed embodiments.

FIG. 1A is a side view of a portion of a shaft of an endoscope including a medical device, according to an embodiment.

FIGS. 1B-C are cross-sectional views of the medical device of FIG. 1A.

FIG. 2A is a cross-sectional view of a medical device, according to another embodiment.

FIG. 2B is a top view of the barrier of FIG. 2A.

FIG. 3A is a cross-sectional view of a medical device, according to another embodiment.

FIG. 3B is a top perspective view of the barrier of FIG. 3A over the intermediary barrier of FIG. 3A.

FIG. 4A is a side view of a medical device, according to another embodiment.

FIG. 4B is a top view of the cartridge of FIG. 4A.

### DETAILED DESCRIPTION

Reference will now be made in detail to aspects of the present disclosure, examples of which are illustrated in the accompanying drawings. Wherever possible, the same or similar reference numbers will be used through the drawings to refer to the same or like parts. The term “distal” refers to a portion farthest away from a user when introducing a device into a subject (e.g., patient). By contrast, the term “proximal” refers to a portion closest to the user when placing the device into the subject.

Both the foregoing general description and the following detailed description are exemplary and explanatory only and are not restrictive of the features, as claimed. As used herein, the terms “comprises,” “comprising,” “having,” “including,” or other variations thereof, are intended to cover a non-exclusive inclusion such that a process, method, article, or apparatus that comprises a list of elements does not include only those elements, but may include other elements not expressly listed or inherent to such a process, method, article, or apparatus. In this disclosure, relative terms, such as, for example, “about,” “substantially,” “generally,” and “approximately” are used to indicate a possible variation of ±10% in a stated value or characteristic.

The present disclosure may solve one or more of the limitations in the art. The scope of the disclosure, however, is defined by the attached claims and not the ability to solve a specific problem. The present disclosure is drawn to medical devices configured to administer doses of agents, e.g., therapeutic agents, among other aspects. The agent may be in any suitable form, including a powder form, which may be delivered to a stream of propellant/pressurized gas, e.g., CO₂, nitrogen, air, etc. Said medical devices allow for the administration of agents in metered doses, which allows for a greater consistency in the quantity of the agent that reaches a target site.

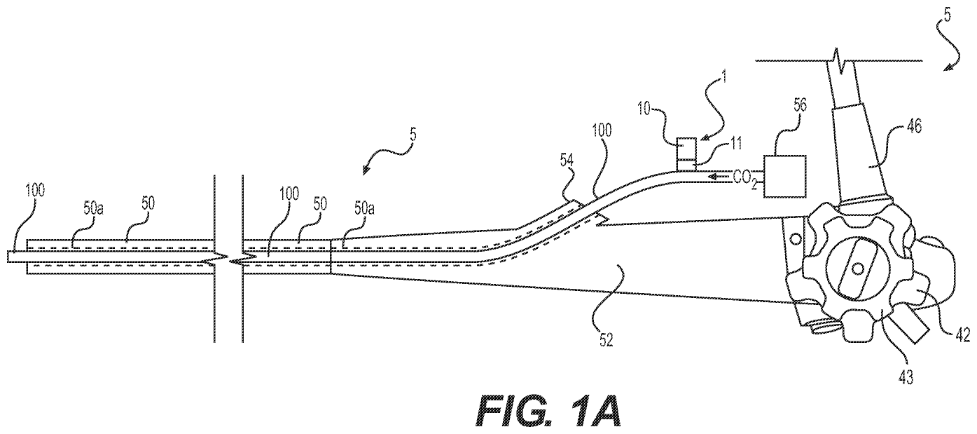

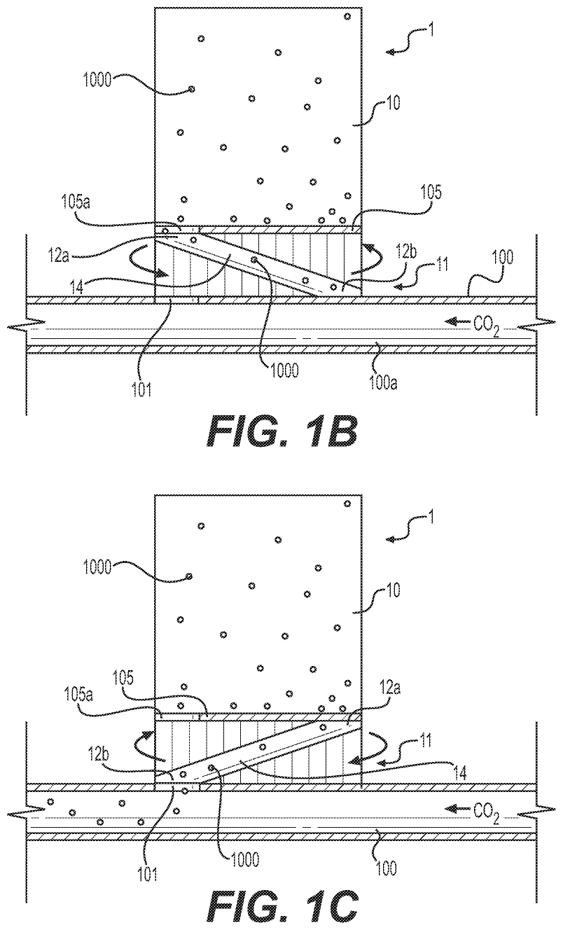

Referring to FIG. 1A, a medical system 5, e.g., an endoscope, according to an embodiment is shown. Medical system 5 includes a flexible shaft 50 (e.g., a catheter) and a handle 52 connected at a proximal end of flexible shaft 50. Handle 52, or some other device for actuating or controlling medical system 5 and any tool or devices associated with medical system 5, includes first and second actuating devices 42, 43, which control articulation of flexible shaft 50, and/or an articulation joint at a distal end of flexible shaft 50, in multiple directions. Devices 42, 43, may be, for example, rotatable knobs that rotate about their axes to push/pull actuating elements (not shown). The actuating elements, such as cables or wires suitable for medical procedures (e.g., medical grade plastic or metal), extend distally from a proximal end of medical system 5 and connect to flexible shaft 50 to control movement thereof. Alternatively, or additionally, a user may operate actuating elements independently of handle 52. Distal ends of actuating elements may extend through flexible shaft 50 and terminate at an actuating joint and/or a distal tip of flexible shaft 50. For example, one or more actuating elements may be connected to an articulation joint, and actuation of actuating elements may control the actuating joint or the distal end of flexible shaft 50 to move in multiple directions.

In addition, one or more electrical cables (not shown) may extend from the proximal end of endoscope 5 to the distal end of flexible shaft 50 and may provide electrical controls to imaging, lighting, and/or other electrical devices at the distal end of flexible shaft 50, and may carry imaging signals from the distal end of flexible shaft 50 proximally to be processed and/or displayed on a display. Handle 52 may also include ports 54, 46 for introducing and/or removing tools, fluids, or other materials from the patient. Port 54 may be used to introduce tools. Port 46 may be connected to an umbilicus for introducing fluid, suction, and/or wiring for electronic components. For example, as shown in FIG. 1A, port 54 receives a tube 100, which extends from the proximal end to the distal end of flexible shaft 50, via a working channel 50a of shaft 50.

As shown in FIG. 1A, tube 100 of medical device 1 is attached to a pressurized gas source 56, e.g., CO₂, which may be controlled by a user to turn on/off and to adjust a rate at which gas flows into tube 100. Source 56 may be a gas canister or tank, a source of gas supplied by a medical facility, or any other suitable source. Medical device 1 further includes an enclosure 10, and a barrier 11 positioned between enclosure 10 and tube 100. Enclosure 10 and barrier 11 are coupled to a proximal portion of tube 100, distal of the connection between tube 100 and source 56.

FIGS. 1B and 1C illustrate an embodiment of medical device 1, l′ in FIG. 1A in further detail. As discussed above, medical device 1 includes enclosure 10 defining a cavity for containing an agent 1000, a tube (e.g., a catheter or a sheath) 100 defining a lumen 100a receiving pressurized gas, e.g., CO₂, from a proximal end, and barrier 11 positioned between the cavity of enclosure 10 and lumen 100a. The shape or size of enclosure 10 is not particularly limited, and may be any suitable shape or size, including cylindrical. As indicated by the directional arrows in FIG. 1B, barrier 11 is rotatable relative to tube 100 and lumen 100a, e.g., about a central axis of barrier 11. In other embodiments, barrier 11 may be rotatable relative to both tube 100 and enclosure 10. In some other embodiments, enclosure 10 may be rotatable relative to barrier 11 and/or tube 100. Rotation of barrier 11 may be by any suitable action, for example, by hand or by mechanical, electrical, or pneumatic action.

Barrier 11 may be an annular, disk-like structure with openings and a passage therethrough. For example, barrier 11 includes a first opening 12a on the barrier surface (e.g., an upper surface) adjacent to the cavity of enclosure 10, for receiving agent 1000 in a passage 14 that extends through barrier 11. Barrier 11 further includes a second opening 12b on the opposite barrier surface (e.g., a bottom surface) adjacent to tube 100 and lumen 100a, from which agent 1000 may be dispensed into lumen 100a. It is noted that the size and shape of first opening 12a and second opening 12b are not particularly limited, and may be any suitable size or shape. First opening 12a and second opening 12b are located on opposite ends of barrier 11, but are connected via passage 14 extending across the length and thickness of barrier 11. Tube 100 also includes an opening 101 which may or may not be aligned with second opening 12b of barrier 11, depending on the rotational position of barrier 11 relative to tube 100 and lumen 100a. Thus, the rotation of barrier 11 relative to tube 100 may establish fluid communication between opening 12b and lumen 100a for delivering agent 1000 from passage 14 to lumen 100a. Enclosure 10 feeds opening 12a with agent 1000 via gravity, and passage 14 storing agent 1000 feeds lumen 100a with agent 1000 via gravity when second opening 12b and lumen opening 101 are aligned. In other embodiments, agent 1000 may be delivered to opening 12a and/or lumen 100a via other suitable mechanisms. Barrier 11 may also be rotated so that second opening 12b and opening 101 of lumen 100a are not aligned, thereby inhibiting the delivery of agent 1000 from passage 14 to lumen 100a. In this instance, passage 14 receives and stores agent 10000, until fluid communication between opening 12b of passage 14 and lumen 100a is established. It is noted that enclosure 10, in any rotational position of barrier 11, is not in fluid communication with lumen 100a. Furthermore, in embodiments prior to any use, passage 14 may be empty an without agent 1000.

In some embodiments, a bottom end of the cavity of enclosure 10 may include a wall 105 adjacent to barrier 11. Wall 105 may include an opening 105a that may or may not be aligned with opening 12a of barrier 11, depending on the rotational position of barrier 11 relative to enclosure 10. Thus, in such embodiments, barrier 11 and/or enclosure 10 may be rotated to align opening 105a with opening 12a of barrier 11 to deliver agent 1000 from enclosure 10 to passage 14 through opening 12a. This is illustrated in FIG. 1B, in which opening 105a of wall 105 and opening 12a of barrier 11 are aligned, thereby feeding passage 14 with agent 1000 from enclosure 10. FIG. 1C shows barrier 11 rotated by approximately 180° from its position in FIG. 1B relative to enclosure 10, and as a result, opening 105a of wall 105 and opening 12a are not aligned, being on opposite ends from one another. It is understood that the barrier 11 may be rotatable to any degree for alignment with opening 12a. Thus, opening 12a is sealed by wall 105, and agent 1000 is no longer fed into opening 12a. It is noted that opening 105a aligns with opening 12a when second opening 12b does not align with opening 101 of tube 100, and opening 105a does not align with opening 12a when second opening 12b aligns with opening 101 of tube 100. Thus, passage 14 may receive agent 1000, prior to agent 1000 being fed to lumen 100a. This allows for medical device 1 to administer a metered dose, i.e., the amount of agent 1000 stored in passage 14, per each degree of rotation, e.g., 180°, of barrier 11 and/or enclosure 10. Furthermore, in some other embodiments, both wall 105 and tube 100 may respectively include a plurality of openings. It is noted that passage 14 is capable of connecting openings of wall 105 and of tube 100 that are 180° apart. However, this is not desired, and in such embodiments, none of the openings of wall 105 are 180° apart from any of the openings of tube 100. As a result, there cannot be fluid communication between passage 14 and said openings of wall 105 and tube 100, at the same time. Instead, fluid communication between passage 14 and the openings of wall 105 and tube 100 is staggered, but not simultaneous. Such embodiments would allow for continuous rotation (both clockwise and counterclockwise) of barrier 11 relative to enclosure 10 to result in passage 14 receiving agent 1000 after a degree of rotation and subsequently dispensing agent 1000 after a further degree of rotation of barrier 11.

Referring to FIGS. 1A-1C, an example of how medical device 1 may be used is further discussed below. A user may deliver a distal end of tube 100 of medical device 1 into the body of a subject, e.g., via a natural orifice (such as a mouth or anus) and through a tortuous natural body lumen of the subject, such as an esophagus, stomach, colon, etc. Tube 100 may be delivered in any suitable way, for example, through working channel 50a of endoscope 5, by inserting a distal end of tube 100 into port 54 of endoscope 5. A user may direct/position the distal end of tube 100 to an intended target site for administration of agent 1000. A user may then fill enclosure 10 with agent 1000, if not filled already, and rotate barrier 11 and/or enclosure 10 relative to tube 100 and lumen 100a so that opening 105a aligns with opening 12a, thereby feeding passage 14 with agent 1000. As discussed above, when opening 105a aligns with opening 12a, second opening 12b does not align with opening 101 of tube 100. Thus, agent 1000 is received and stored by passage 14. A user may then rotate barrier 11 to align opening 12b with opening 101 of tube 100, so that all of agent 1000 stored in passage 14 is fed from passage 14 to lumen 100a, thereby administering a metered dose of agent 1000. A user may turn on the pressurized gas source at any time prior to the alignment of opening 12b with opening 101 and supply pressurized gas until the metered dose of agent 1000 reaches the target tissue site. Alternatively, a user may start supply of pressurized gas after the supply of agent 1000 to lumen 100a.

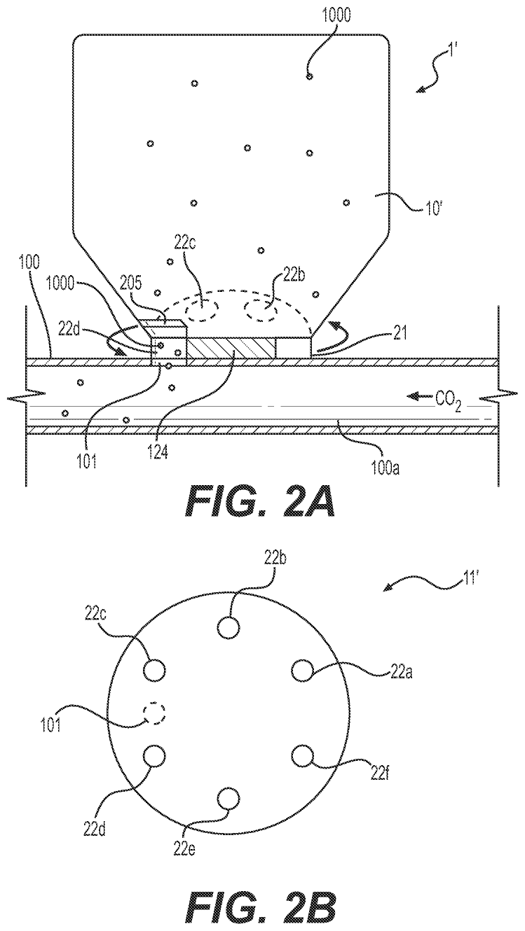

In FIG. 2A, another embodiment of medical device 1′ is shown. Like the embodiment discussed above, medical device 1′ includes an enclosure 10′ defining a cavity for containing an agent 1000, a tube (e.g., a catheter or a sheath) 100 defining a lumen 100a receiving pressurized gas, e.g., CO₂, from a proximal end, and a barrier 21 positioned between the cavity of enclosure 10 and lumen 100a. The shape or size of enclosure 10′ is also not particularly limited, and may be any suitable shape or size. As indicated by the directional arrows in FIG. 2A, barrier 21 is also rotatable relative to tube 100 and lumen 100a. In other embodiments, barrier 21 may be rotatable relative to both tube 100 and enclosure 10′. In some other embodiments, enclosure 10′ may be rotatable relative to barrier 21 and/or tube 100. Rotation of barrier 21 and enclosure 10′ may also be actuated by any suitable action.

Barrier 21, as shown in both FIGS. 2A and 2B, includes a plurality of openings, i.e., six openings 22a-22f, of equal or approximately equal size, i.e., width or diameter, symmetrically arranged radially about a central axis of rotation of barrier 21. It is noted that the number of openings, the size of openings, the shape of openings, and the arrangement of openings on barrier 21 is not particularly limited, and may be any suitable configuration. For example, in other embodiments, barrier 21 may have four circular openings, each of which has varying diameters from one another. Each of openings 22a-22f extends through the thickness of barrier 21, and is configured to receive and store a pre-determined or selected amount of agent 1000, depending on the size of the openings. Thus, agent 1000 from enclosure 10 feeds into openings 22a-22f, via gravity in some embodiments, until said openings are filled. Note that FIG. 2A shows half of barrier 21 in perspective to show the position of openings 21a, 21b, 21c, and 21d.

By rotation of barrier 21 relative to tube 100 and lumen 100a, one of openings 22a-22f may align with opening 101, thereby establishing fluid communication between the one opening and lumen 100a for delivering agent 1000 from the one opening to lumen 100a via gravity. In some embodiments, enclosure 10′ may further include a seal 205, which is positioned adjacently above barrier 21, above where one of openings 22a-22f may be located, and directly above opening 101 of tube 100. Thus, as one opening of openings 22a-22f aligns with opening 101 via rotation of barrier 21, an excess amount of agent 1000 above the one opening is shaved off by seal 205 and the one opening is sealed from receiving further agent 1000 from enclosure 10′ when that opening aligns with opening 101 of tube 100. This allows for medical device 1′ to administer a metered dose, i.e., the amount of agent 1000 stored in openings 22a-22f, per each degree of rotation, e.g., 60°, of barrier 21 and/or enclosure 10′. It is noted that as a result of such configuration, when fluid communication is established between one of openings 22a-22f and lumen 100a, no fluid communication is established between the other remaining openings and lumen 100a, as the bottom of the remaining openings is sealed by tube 100.

As shown in FIG. 2B, which shows a top view of barrier 21, barrier 21 may also be rotated so that none of openings 22a-22f are aligned with opening 101 of tube 100, thereby inhibiting the delivery of agent 1000 from any of openings 22a-22f to lumen 100a. In this instance, an amount of agent 1000 is stored in openings 22a-22f until fluid communication between the openings and lumen 100a is established.

Referring to FIGS. 2A-2B, an example of how medical device 1′ may be used is further discussed below. Similar to medical device 1, medical device 1′ may be delivered into the body of a subject, and directed to an intended target site for agent 1000 administration in the same manner. A user may then fill enclosure 10′ with agent 1000, if not filled already, which will fill openings 22a-22f with agent 1000. The user then may rotate barrier 21 relative to tube 100 and lumen 100a so that one of openings 22a-22f aligns with opening 101. Such alignment results in seal 205 shaving off an excess amount of agent 1000 above the one opening, sealing the one opening from being fed any more of agent 1000 from enclosure 10′, and feeding lumen 100a with agent 1000 stored in the one opening. As discussed above, when the one opening aligns with opening 101 of tube 100, the remaining openings 22a-22f do not align with opening 101 of tube 100. Thus, agent 1000 is stored within the remaining openings 22a-22f, until each of the remaining openings is aligned with opening 101 via rotation of barrier 21, in turn. A user may turn on the pressurized gas source at any time prior to or during the alignment of one of the openings 22a-22f with opening 101, as in the embodiment described in connection with FIGS. 1B-1C.

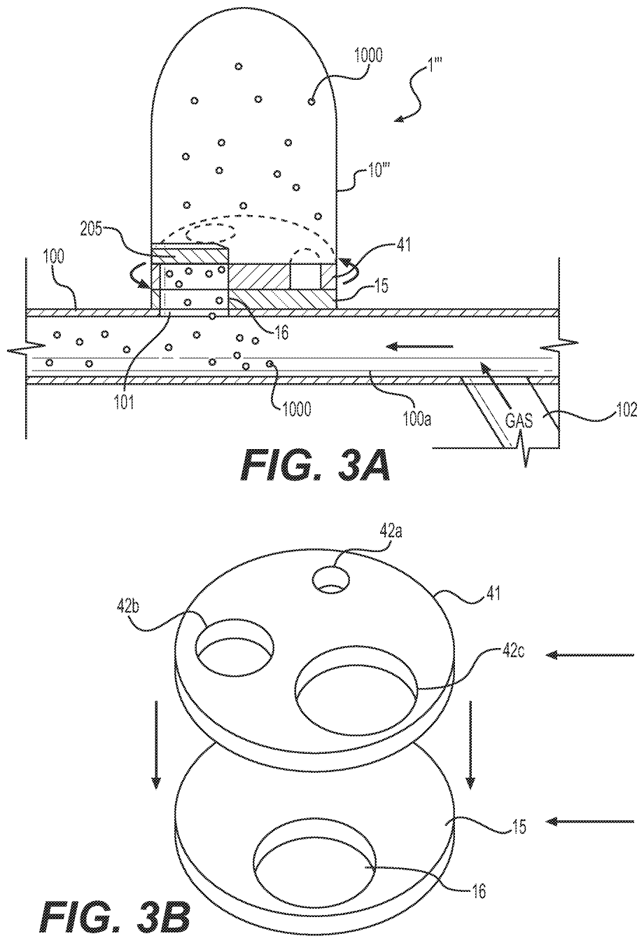

In FIG. 3A, another embodiment of medical device 1′″ is shown. Medical device 1′″ is similar to medical device 1′, and differences between the two devices will be highlighted. Device 1′″ may include any of the features of device 1′ and operate in the same or substantially the same way. Medical device 1′″ includes an enclosure 10′″, a barrier 41, and lumen 100. Moreover, enclosure 10′″ further includes a seal 205, which is positioned adjacently above barrier 41, above where one of openings 42a-42c may be located, and directly above opening 101 of tube 100. However, unlike medical device 1′, lumen 100a of medical device 1′″ receives pressurized gas from a second lumen 102, which is connected to tube 100 at a point that is proximal to opening 101. Alternatively, medical device 1′″ may receive pressurized gas from a proximal end of lumen 100a, and may be without second lumen 102. Medical device 1′″ further includes, in at least some embodiments, an intermediary barrier 15 including an opening 16, positioned between barrier 41 and tube 100. As indicated by the directional arrows in FIG. 3A, barrier 41 is rotatable relative to intermediary barrier 15, tube 100, and lumen 100a. In other embodiments, enclosure 10′″ may also be rotatable relative to barrier 41, intermediary barrier 15, tube 100, and lumen 100a. Rotation of enclosure 10′″ and barrier 41 may be actuated by any suitable action.

Barrier 41, as shown in both FIGS. 3A and 3B, includes three openings, i.e., 42a-42c, of different sizes, i.e., widths or diameter, arranged radially about a central axis of rotation of barrier 41, like barrier 21 (referring to FIG. 2B). A first opening 42a has the smallest width of the three openings, a second opening 42c has the largest width of the three openings, and a third opening 42b has a width in between that of first opening 42a and that of second opening 42c. Thus, each of openings 42a-42c receives and stores different amounts or doses of agent 1000.

To help a user differentiate between the different sizes of openings 42a-42c, enclosure 10′″ and/or barrier 41 may further include markings on their outer surfaces that indicate the locations of openings 42a-42c relative to one another, and to openings 16 and 101. Thus, a user may rotate barrier 41 and/or enclosure 10′″, relative to intermediary barrier 15, tube 100, and lumen 100a, to select one of openings 42a-42c based on a desired amount or dose of agent 1000.

Intermediary barrier 15, as shown in both FIGS. 3A and 3B, includes opening 16. Opening 16 may be aligned with lumen opening 101, and also openings 42a-42c, depending on the rotational position of barrier 41 relative to intermediary barrier 15. As shown in FIG. 3B, opening 16 is at least the same width as the largest opening of barrier 41, i.e., second opening 42c.

Any one of openings 42a-42c may be aligned with intermediary opening 16 and lumen opening 101 via rotation of barrier 21. Such alignment establishes fluid communication between one of openings 42a-42c and lumen 100a for delivering agent 1000 from one of openings 42a-42c to lumen 100a via gravity. Similar to that of medical device 1′, as one opening of openings 42a-42c aligns with opening 16 of intermediary barrier 15 and opening 101 via rotation of barrier 41, an excess amount of agent 1000 above the one opening is shaved off by seal 205 and the one opening is sealed from further receiving agent 1000 from enclosure 10″. This allows for medical device 1′″ to administer a metered dose, the amount of agent 1000 stored in openings 42a-42c, per each degree of rotation, e.g., 120°, of barrier 41. As a result of such configuration, when fluid communication is established between one of openings 42a-42c and lumen 100a, no fluid communication is established between the other remaining openings 42a-42c and lumen 100a.

Barrier 41 may also be rotated so that none of openings 42a-42c is aligned with intermediary opening 16 and lumen opening 101, thereby inhibiting the delivery of agent 1000 from enclosure 10′″ to lumen 100a. In this instance, varying amounts of agent 1000 are stored in openings 42a-42c until fluid communication between openings 42a-42c and lumen 100a is established.

It is further noted in some embodiments, intermediary barrier 15 may also be rotatable relative to enclosure 10′″, barrier 41, tube 100, and lumen 100a, so that opening 16 does not align with any of the openings of barrier 41, and/or opening 101 as well. This is applicable in embodiments having barriers with multiple openings. By being able to misalign opening 16 from opening 101, a user may select another opening 42a, 42b, 42c, etc., via rotation of barrier 41, that is not adjacent to the currently aligned opening, without having to inadvertently dispense agent 1000 stored in openings adjacent to the currently aligned opening, via the necessary degree of rotation to select other non-adjacent openings.

Referring to FIGS. 3A-3B, an example of how medical device 1′″ may be used is further discussed below. Similar to medical devices 1 and 1′, medical device 1′″ may be delivered into the body of a subject, and directed to an intended target site for agent 1000 administration in the same manner. A user may then fill enclosure 10′ with agent 1000, if not filled already, and rotate barrier 41 relative to intermediary barrier 15, tube 100, and lumen 100a, so that one of openings 42a-42c aligns with openings 16 and 101. Such alignment results in seal 205 shaving off an excess amount of agent 1000 above the one opening, sealing the one opening from being fed any more of agent 1000 from enclosure 10′″, and feeding lumen 100a with agent 1000 stored in the one opening. As discussed above, when the one opening aligns with opening 16 of intermediary barrier 15 and opening 101 of tube 100, the remaining openings 42a-42c do not align with openings 16 and 101. Thus, agent 1000 is stored within the remaining openings 42a-42c, until each of the remaining openings is aligned with openings 16 and 101 via rotation of barrier 41. A user may turn on the pressurized gas source at any time prior to or during the alignment of one of the openings 42a-42c with openings 16 and 101, as in previously described embodiments.

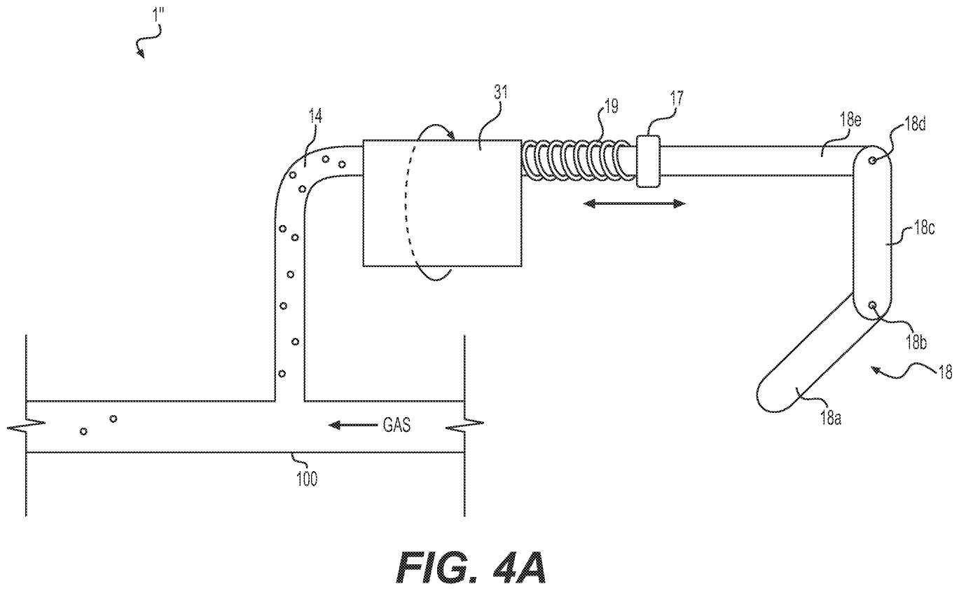

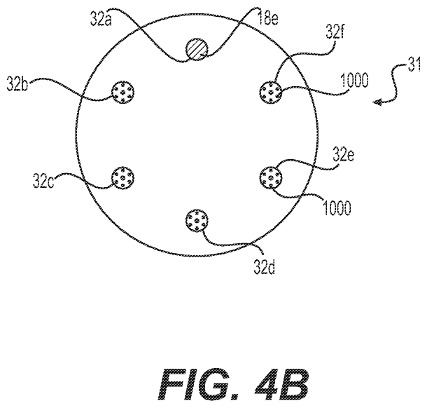

Referring to FIG. 4A, another embodiment of medical device 1″ is shown. Medical device 1″ includes a trigger 18, a rotating cartridge 31 including a plurality of chambers, a lumen 100 receiving pressurized gas from a proximal end, and a channel 14 establishing fluid communication between a distal end of cartridge 31 and lumen 100 for delivering agent 1000 from cartridge 31 to lumen 100. As shown in FIG. 4B, cartridge 31 includes a plurality of symmetrically-arranged chambers, i.e., six chambers 32a-32f, of equal or substantially equal size, each of which stores a pre-filled amount of agent 1000.

Trigger 18 includes a lever 18a coupled to a linkage 18c via an articulating joint 18b, and linkage 18c coupled to a plunger 18e via another articulating joint 18d. A distal portion of plunger 18e is housed within a proximal portion of cartridge 31, and is coupled to cartridge 31 in any suitable manner so that the distal end of plunger 18e faces one of chambers 32a-32f with which plunger 18e is aligned. A spring 19 coils around a distal portion of plunger 18e outside of cartridge 31 up until a stop 17 fixated on plunger 18e, thereby spring-biasing plunger 18e in its aforementioned position of facing one of chambers 32a-32f. Spring 19 is not particularly limited and may be any suitable spring. Likewise, stop 17 may be of any suitable material, such as rubber.

Trigger 18 is configured so that when lever 18a is pulled proximally, linkage 18c likewise pivots proximally relative to plunger 18e via articulating joint 18d. Such movements of lever 18a and linkage 18c result in plunger 18e longitudinally advancing towards cartridge 31 and into one of chambers 32a-32f, thereby propelling the pre-filled amount of agent 1000 towards and through channel 14, which extends downward to tube 100, thereby feeding agent 1000 to tube 100 via gravity. The longitudinal advancement of plunger 18e may be actuated by any suitable mechanisms, including, but not limited to, mechanical, electrical, or pneumatic mechanisms. Plunger 18e advances within cartridge 31 and one of chambers 32a-32f up until spring 19 is fully compressed, thereby inhibiting any further advancement of plunger 18e towards cartridge 31. Once lever 18 is released, spring-biased plunger 18e automatically reverts back to its initial position of being outside of and facing one of chambers 32a-32f.

Cartridge 31 is rotatable relative to plunger 18e so that any one of chambers 32a-32f is aligned with plunger 18e. In some embodiments, cartridge 31 may be configured to rotate or revolve automatically, after one of chambers 32a-32f is emptied by plunger 18e, so that an adjacent chamber 32a-32f storing a pre-filled amount of agent 1000 is aligned with plunger 18e.

It will be apparent to those skilled in the art that various modifications and variations can be made to the disclosed device without departing from the scope of the disclosure. Other embodiments of the disclosure will be apparent to those skilled in the art from consideration of the specification and practice of the invention disclosed herein. It is intended that the specification and examples be considered as exemplary only, with a true scope and spirit of the invention being indicated by the following claims.

1-20. (canceled)

21. A medical system, comprising:
an endoscope including a shaft;
a pressurized fluid source in fluid communication with the endoscope, the pressurized fluid source is configured to selectively deliver a pressurized fluid towards the endoscope; and
a medical device in fluid communication with the endoscope, the medical device including an enclosure storing an agent and a barrier configured to selectively deliver the agent out of the enclosure and towards the endoscope;
wherein the medical system is configured such that the pressurized fluid from the pressurized fluid source and the agent from the medical device are mixed with one another prior to entering the endoscope, and the endoscope is configured to receive and deliver a mixture of the pressurized fluid and the agent through the shaft.

22. The medical system of claim 21, further comprising a tube that is coupled to a port of the endoscope and extends through a working channel of the shaft via the port.

23. The medical system of claim 22, wherein the pressurized fluid source and the medical device are fluidly coupled to the endoscope via the tube.

24. The medical system of claim 22, wherein the barrier is configured to move relative to the enclosure to selectively release the agent from the enclosure.

25. The medical system of claim 24, wherein the barrier includes a passage that is positioned between the enclosure and the tube, the passage is configured to transfer the agent stored in the enclosure to the tube in response to the barrier moving relative to the enclosure.

26. The medical system of claim 25, wherein rotation of the barrier relative to the enclosure and the tube establishes fluid communication between a cavity of the enclosure and a lumen of the tube via the passage.

27. The medical system of claim 26, wherein the barrier is configured to align the passage with each of the enclosure and the tube to selectively deliver the agent from the cavity to the lumen in response to rotating the passage.

28. The medical system of claim 25, wherein the enclosure is configured to feed the agent to the passage via gravity, and the passage is configured to feed the agent to the lumen via gravity.

29. The medical system of claim 21, wherein the pressurized fluid source is selectively actuated to initiate delivery of the pressurized fluid to the endoscope independent of the agent from the medical device.

30. The medical system of claim 21, wherein the medical device is selectively actuated to initiate delivery of the agent to the endoscope independent of the pressurized fluid from the pressurized fluid source.

31. The medical system of claim 21, wherein the pressurized fluid source includes a gas canister or tank positioned within a medical facility and adjacent to the endoscope and the medical device.

32. The medical system of claim 22, wherein the barrier includes a plurality of openings positioned between the enclosure and the tube, the plurality of openings are each configured to transfer the agent stored in the enclosure to the tube in response to the barrier moving relative to the enclosure.

33. A medical system, comprising:
an endoscope including a shaft;
a pressurized fluid source positioned external to and in fluid communication with the endoscope, the pressurized fluid source is selectively actuated to release a pressurized fluid; and
a medical device positioned external to and in fluid communication with the endoscope, the medical device is selectively actuated to release an agent;
wherein each of the pressurized fluid source and the medical device are selectively actuated independent of one another;
wherein the pressurized fluid released from the pressurized fluid source is configured to mix with the agent released from the medical device prior to entering the endoscope, and urge a mixture of the pressurized fluid and the agent into the endoscope and through the shaft.

34. The medical system of claim 33, further comprising a tube that is coupled to a port of the endoscope and extends through a working channel of the endoscope, wherein the pressurized fluid source and the medical device are fluidly coupled to the endoscope via the tube.

35. The medical system of claim 34, wherein the medical device includes an enclosure configured to store the agent and a barrier configured to release the agent from the enclosure in response to the barrier rotating relative to the enclosure.

36. The medical system of claim 35, wherein the barrier is configured to release the agent towards the endoscope in response to the barrier rotating relative to the enclosure.

37. The medical system of claim 35, wherein the barrier includes one or more openings defining a passage that is positioned between the enclosure and the tube, the one or more openings are configured to transfer the agent stored in the enclosure to the tube in response to the barrier rotating relative to the enclosure.

38. A method of administering an agent via a medical system that includes an endoscope, a pressurized fluid source, and a medical device, the method comprising:
coupling the pressurized fluid source with a tube;
separate from the step of coupling the pressurized fluid source with the tube, coupling the medical device with the tube;
coupling the tube with the endoscope to establish fluid communication between (1) the pressurized fluid source and the medical device, with (2) the endoscope, via the tube;
actuating the pressurized fluid source to deliver a pressurized fluid through the tube;
separate from the step of actuating the pressurized fluid source, actuating the medical device to deliver an agent through the tube, wherein the medical system is configured such that the pressurized fluid from the pressurized fluid source and the agent from the medical device are mixed with one another within the tube prior to entering the endoscope; and
receiving a mixture of the pressurized fluid and the agent at the endoscope and delivering the mixture through a shaft of the endoscope.

39. The method of claim 38, wherein actuating the medical device to deliver the agent through the tube comprises:
rotating a barrier of the medical device to selectively align a passage of the barrier with each of an enclosure of the medical device that stores the agent and a lumen of the tube that receives the agent from the enclosure via the passage.

40. The method of claim 38, wherein coupling the tube with the endoscope comprises:
extending the tube into a port of the endoscope and through a shaft of the endoscope that terminates at a target treatment site, such that the mixture of the pressurized fluid and the agent are delivered through the shaft and to the target treatment site.
